(19) United States
(12) Patent Application Publication
(10) Pub. No.: US 2025/0275712 A1
(43) Pub. Date: Sep. 4, 2025

(54) BREATH TEST METHOD AND TEST MEAL

(71) Applicant: Advanced Breath Diagnostics, LLC, Brentwood, TN (US)

(72) Inventors: Ethan S. McClain, Franklin, TN (US); Nicole L. Giffin, Gallatin, TN (US); Catherine E. Williams, Fairview, TN (US); Kerry C. Bush, Franklin, TN (US)

(21) Appl. No.: 19/065,055

(22) Filed: Feb. 27, 2025

Related U.S. Application Data
(60) Provisional application No. 63/559,637, filed on Feb. 29, 2024.

(51) Int. Cl. A61B 5/00; G01N 33/497

(52) U.S. Cl. CPC A61B 5/4238; G01N 33/497

(57) ABSTRACT

A breath test method is provided that includes the steps of (a) obtaining a pre-meal baseline breath sample from a test subject, (b) providing a test meal comprising oatmeal and ¹³C-Spirulina, the ¹³C-Spirulina being bound to components of the oatmeal, (c) allowing the test subject to consume the test meal, (d) obtaining breath samples from the test subject at a series of time points after consumption of the test meal, and (e) analyzing the breath samples.

### RELATED APPLICATION

The present application claims priority to U.S. provisional application No. 63/559,637, the entire contents of which is incorporated by reference.

### FIELD OF THE INVENTION

This invention relates generally to a test meal including an edible food component that includes a marker. The invention also relates to gastric emptying breath tests for evaluating gastric emptying rates in test subjects.

### BACKGROUND OF THE INVENTION

Gastroparesis (or delayed gastric emptying) is a condition that affects motility in the stomach and causes food to stay in the stomach for too long. Normally, the stomach muscles contract to propel food through the digestive tract, moving food from the stomach to the small intestine. However, in a subject with gastroparesis, the stomach muscles do not contract normally, thereby causing a delay in the rate of gastric emptying. Gastroparesis can be diagnosed by evaluating a gastric emptying rate in a subject. Currently, there are two types of tests for measuring gastric emptying rate: gastric scintigraphy tests and breath tests.

In a scintigraphy test, a subject ingests a test meal including a component that has been radiolabeled with a radioisotope. The gamma emission from the radiolabel is measured by a scintillation camera it moves from the stomach to the small intestine. Several disadvantages are associated with the scintigraphy test. First, subjects must be subjected to radioisotopes. This is particularly problematic for women of childbearing age or children. Further, the test must be carried out at specialized nuclear medicine facilities. Scintigraphy tests cannot be administered in a regular clinical setting or at home through virtual telehealth due to radiation risks and requirements for specially licensed personnel and facilities to handle radioactive materials.

In a breath test, a subject injests a test meal including a component that has been labeled with a non-radioactive stable isotope. While a number of breath tests have been developed, there is currently only one standardized, FDA-approved gastric emptying breath test (“GEBT method”) for evaluating a gastric emptying rate in a subject. The GEBT method is disclosed in PMA No. P110015 and approved by the FDA on Apr. 6, 2015. This standardized GEBT method uses an egg-based test meal that contains reconstituted lyophilized scrambled egg mix having components bound to ¹³C-Spirulina (“egg-based test meal”). The egg-based test meal also includes Nabisco PREMIUM saltine crackers.

One drawback with the egg-based test meal is that it includes whole eggs. Some subjects may have an allergy to whole eggs and therefore be ineligible to use this test. Further, the incidence of allergies to whole eggs is higher in infants and children and it is often unknown whether the infant or child has an allergy or intolerance to whole eggs. If such an unknown allergy or intolerance exits, it may have allergenic risks to the patient and/or it may impact the test results.

It would therefore be desirable to provide an alternative breath test meal to the egg-based test meal that does not include eggs. It would also be desirable to be able to use such an alternative breath test meal in the standardized, FDA-approved GEBT method. It would also be desirable to provide an option to elect between different tests meals for use in the GEBT method.

### BRIEF DESCRIPTION OF THE DRAWINGS

FIG. 1 is a graph showing ¹³CO₂ excretion rates at a series of time points for healthy subjects administered an oatmeal-based meal.

FIG. 2 is a graph showing ¹³CO₂ excretion rates at a series of time points for healthy subjects administered a standardized egg-based test meal.

FIG. 3 is a graph showing average ¹³CO₂ excretion rates at a series of time points for healthy subjects administered an oatmeal-based meal and a standardized egg-based test meal.

### SUMMARY

Certain embodiments provide a gastric emptying breath test meal. In some cases, the breath test meal includes oatmeal and ¹³C-Spirulina, and the ¹³C-Spirulina is bound to components of the oatmeal. In some cases, the oatmeal comprises 100% whole grain oats. In certain cases, the oatmeal consists essentially of 100% whole grain oats. In specific cases, the oatmeal comprises Quaker Oats. In one example, the oatmeal comprises Quaker Quick Oats. Quaker Oats and Quaker Quick Oaks are products obtained from The Quaker Oats Company.

The ¹³C-Spirulina can be present in an amount from 50 to 200 mg, such as from 75 mg to 125 mg, from 95 mg to 105 mg or from 99 mg to 101 mg. In certain cases, the ¹³C-Spirulina can be present in an amount of 100 mg. The oatmeal can be present in an amount of from 20 grams to 60 grams, such as from 30 grams to 50 grams, from 35 grams to 45 grams, or from 39 grams to 41 grams. In specific cases, the oatmeal is present in an amount of 40 grams.

In some cases, the breath test meal also includes whole milk. The whole milk can be present in an amount of from 90 mL to 270 mL, such as from 150 mL to 200 mL, from 170 mL to 180 mL, or from 176 mL to 178 mL. In certain cases, the whole milk is present in an amount of 177 mL.

In further cases, the breath test meal also includes cane sugar. The cane sugar can be present in an amount of from 8 grams to 20 grams, such as from 10 grams to 18 grams, from 12 grams to 16 grams, or from 13 grams to 15 grams. In certain cases, the cane sugar is present in an amount of 14 grams.

In even further cases, the breath test meal can also include water. The water can be present in an amount of from 120 mL to 240 mL, such as from 160 mL to 200 mL, from 170 mL to 190 mL, from 175 mL to 185 mL, or from 179 mL to 181 mL. In certain cases, the water is present in an amount of 180 mL.

In some cases, the breath test meal includes oatmeal, whole milk, cane sugar and ¹³C-Spirulina. Each component can be present in any range described herein. In some cases, the oatmeal is present in an amount of from 20 grams to 60 grams, the whole milk is present in an amount of from 90 mL to 270 mL, the cane sugar is present in an amount of from 8 grams to 20 grams, and the ¹³C-Spirulina is present in an amount of from 50 mg to 200 mg.

In additional cases, the breath test meal includes oatmeal, whole milk, cane sugar, ¹³C-Spirulina and water. Each component can be present in any range described herein. In some cases, the oatmeal is present in an amount of from 20 grams to 60 grams, the whole milk is present in an amount of from 90 mL to 270 mL, the cane sugar is present in an amount of from 8 grams to 20 grams, the ¹³C-Spirulina is present in an amount of from 50 mg to 200 mg, and the water is present in an amount of from 120 mL to 240 mL.

Other embodiments provide a breath test method. The method can include the steps of (a) obtaining a pre-meal baseline breath sample from a test subject, (b) providing a breath test meal comprising oatmeal and ¹³C-Spirulina, the ¹³C-Spirulina being bound to components of the oatmeal, (c) allowing the test subject to consume the breath test meal, (d) obtaining breath samples from the test subject at a series of time points after consumption of the breath test meal, and (e) analyzing the breath samples. The breath test meal can include any breath test meal described herein.

In some cases, the series of time points includes 45, 90, 120, 150, 180 and 240 minute time points. In certain cases, the series of time points can also include a 15 minute time point, a 30 minute time point, a 45 minute time point, a 60 minute time point and/or a 210 minute time point. In specific cases, the series of time points can include 15, 30, 45, 60, 90, 120, 150, 180, 210 and 240 minute time points.

Also, in some cases, the step of obtaining a pre-meal baseline breath sample takes place after the test subject fasts for at least 8 hours. Further, in some cases, the step of allowing the test subject to consume the breath test meal includes prompting the test subject to consume the breath test meal within 10 minutes. Even further, in some cases, the step of collecting the breath test samples from the test subject includes depositing breath samples into collection tubes and then capping the collection tubes. Finally, in some cases, the step of analyzing the breath samples can include using Gas Isotope Ratio Mass Spectrometry to determine the ratio of ¹³CO₂/¹²CO₂ in each breath sample.

### DETAILED DESCRIPTION OF PREFERRED EMBODIMENTS

The following detailed description is to be read with reference to the drawings, in which like elements in different drawings have like reference numerals. The drawings, which are not necessarily to scale, depict selected embodiments and are not intended to limit the scope of the invention. Skilled artisans will recognize that the examples provided herein have many useful alternatives that fall within the scope of the invention.

Applicant has developed an alternative test meal that can be used in the standardized, FDA-approved gastric emptying breath test (“GEBT method”) disclosed in PMA No. P110015 and approved by the FDA on Apr. 6, 2015, the entire contents of which are incorporated herein by reference.

The GEBT method currently uses an egg-based test meal consisting of 27 grams of re-hydrated, pasteurized scrambled egg mix containing a dose of 43 mg of ¹³C (provided by approximately 100 mg of ¹³C-Spirulina), 6 saltine crackers, and 180 mL of water. The pasteurized scrambled egg mix includes whole eggs, water, nonfat dry milk, salt, and smoke flavoring. The ¹³C-Spirulina is cultured in a >95% ¹³C enriched medium, resulting in full ¹³C-labeling of nearly all of the algae's carbon containing nutrients.

Applicant has discovered a new test meal that can be used in the standardized GEBT method as an alternative to the egg-based test meal. The new test meal comprises oatmeal and ¹³C-Spirulina, wherein the ¹³C-Spirulina is bound to components of the oatmeal (“oatmeal-based test meal”). In some cases, the oatmeal-based test meal also includes whole milk, cane sugar and water. The ¹³C-Spirulina in the oatmeal-based test meal can also be cultured in a >95% ¹³C enriched medium, resulting in full ¹³C-labeling of nearly all of the algae's carbon containing nutrients. The oatmeal-based test meal can be prepared by combining the contents and microwaving the contents until a desired thickness is obtained. In some cases, oatmeal-based test meal components can be microwaved on 50% power for 3 minutes, stirred, and microwaved in 1-minute increments until thick.

When the oatmeal-based test meal is administered according to the standardized GEBT method, it surprisingly provides results comparable to results obtained by administering the egg-based test meal. This was an unexpected discovery because the oatmeal-based test meal includes a completely different matrix than the egg-based test meal. It was previously believed that whole eggs in the egg-based test meal provided a unique protein/fat matrix that allowed the ¹³C-Spirulina to remain bound to whole egg components while travelling through the subject's digestive tract. However, Applicant surprisingly discovered that ¹³C-Spirulina also remains bound to the matrix of the oatmeal-based test meal while travelling through a digestive tract.

The oatmeal-based test meal is beneficial because it does not include eggs and therefore can be used in infants and children and in subjects known to have an egg intolerance. The oatmeal-based test meal is also advantageous because it can be used in the standardized GEBT method. This provides quite an advantage because clinicians do not need to use an entirely different breath test method to use an alternative oatmeal-based test meal. The oatmeal-based test meal also provides a simple alternative to the egg-based meal, so that test subjects can choose which meal sounds more palatable, or so that a clinician can elect which meal is more suitable for a given subject.

Some embodiments provide a gastric emptying breath test meal comprising oatmeal and ¹³C-Spirulina, wherein the ¹³C-Spirulina is bound to components of the oatmeal. The oatmeal can be provided in an amount of from 20 grams to 60 grams, such as from 30 grams to 50 grams, from 35 grams to 45 grams, or from 39 grams to 41 grams. In specific cases, the oatmeal is provided in an amount of 40 grams. The ¹³C-Spirulina can be provided in an amount from 50 to 200 mg, such as from 75 mg to 125 mg, from 95 mg to 105 mg or from 99 mg to 101 mg. In certain cases, the ¹³C-Spirulina can be provided in an amount of 100 mg.

In some cases, the meal further includes whole milk, which can be provided in an amount of from 90 mL to 270 mL, such as from 150 mL to 200 mL, from 170 mL to 180 mL, or from 176 mL to 178 mL. In certain cases, the whole milk is provided in an amount of 177 mL.

Also, in some cases, the meal further includes cane sugar, which can be provided in an amount of from 8 grams to 20 grams, such as from 10 grams to 18 grams, from 12 grams to 16 grams, or from 13 grams to 15 grams. In certain cases, the cane sugar is provided in an amount of 14 grams.

In additional cases, the meal also includes water, which can be provided in an amount of from 120 mL to 240 mL, such as from 160 mL to 200 mL, from 170 mL to 190 mL, from 175 mL to 185 mL, or from 179 mL to 181 mL. In certain cases, the water is provided in an amount of 180 mL.

Additional embodiments provide a gastric emptying breath test meal comprising oatmeal, whole milk, cane sugar and ¹³C-Spirulina. The oatmeal can be provided in an amount of from 20 to 60 grams, such as 40 grams. The whole milk can be provided in an amount of from 90 mL to 270 mL, such as 177 mL. The ¹³C-Spirulina can be provided in an amount of from 50 mg to 200 mg, such as 100 mg. In other cases, the meal also includes water, which can be provided in an amount of from 120 mL to 240 mL, such as 180 mL.

Other embodiments provide a breath test method using an oatmeal-based test meal. The oatmeal-based test meal can include any embodiment described herein. The method can include steps of (1) collecting a pre-meal fasting breath sample from subject, (2) preparing an oatmeal-based meal, (3) allowing a subject to consume the oatmeal-based test meal, and (4) collecting breath samples at a series of time points after the subject consumes the oatmeal-based test meal, (5) and analyzing the breath samples at each time point. The method can be performed by a test administrator such as a clinician in a clinical setting. In some cases, the method can be performed virtually via telehealth. In some cases, the series of time points include any of 15, 30, 45, 60, 90, 120, 150, 180, 210 and 240 minute time points. In certain cases, the series of time points include the 45, 90, 120, 150, 180 and 240 minute time points. In specific cases, the series of time points include all 15, 30, 45, 60, 90, 120, 150, 180, 210 and 240 minute time points.

One exemplary breath test method using the oatmeal-based test meal will now be described.
1. Prompting a test subject to fast overnight for at least 8 hours. No solid foods or liquids other than 120 mL of water up to 1 hour before the test is allowed.
2. Collecting a pre-meal, baseline breath sample. Any method of collecting breath samples can be used but in preferred cases, the breath samples are deposited into collection tubes and then the collection tubes are capped.
3. Preparing the oatmeal-based meal.
4. Prompting the test subject to ingest the oatmeal-based test meal along 180 mL of water. The test subject is also prompted to consume the oatmeal-based test meal and water within 10 minutes.
5. Collecting a breath sample at 15, 30, 45, 60, 90, 120, 150, 180, 210 and 240 minute time points after the test subject consumes the oatmeal-based test meal.
6. Using a Gas Isotope Ratio Mass Spectrometry to determine the ratio of ¹³CO₂/¹²CO₂ in each breath sample. The ratio is then used to calculate the ¹³CO₂ excretion rate for each breath sample. The rate of ¹³CO₂ excretion at each GEBT measurement time is directly proportional to rate of gastric emptying. Preferably, the excretion rate for each breath sample is reported using a kPCD metric. The acronym kPCD stands for “1000 X Percent Carbon-13 Dose (PCD) excreted per minute.” A kPCD value at each time point (kPCDₜ) is calculated at each GEBT measurement time using the following formula:

$${kPCD}_{t} = [ \frac{DOB \times {CO}_{2} PR \times {R}_{s} \times 13}{10 \times dose} ] \times 1000$$

Where:
   DOB=The measured difference in the ratio [¹³CO₂/¹²CO₂] between a breath sample at a time point and the baseline breath sample.
   CO₂PR=CO₂ production rate (mmol CO₂/min) calculated using Schofield equations, which incorporate the subject's age, sex, height, and weight.
   Rₛ=The ratio [¹³CO₂/¹²CO₂] in a reference standard (Pee Dee belemnite) for these measurements. Rₛ=0.0112372
   13=the atomic weight of Carbon-13
   10=A constant factor for converting units
   dose=the weight (mg) of Carbon-13 in the dose of ¹³C-Spirulina administered to the test subject. Since ¹³C-Spirulina is approximately 43% Carbon-13, a 100 mg dose of ¹³C-Spirulina contains approximately 43 mg of Carbon-13.
7. The kPCD values for each time point (pre-meal, 15, 30, 45, 60, 90, 120, 150, 180, 210 and 240 minute time points) are provided. Preferably, the kPCD values are provided in a graphical display showing the subject's kPCD at each time point. The graphical display can also include a reference range cutoff point at each time point. The cutoff point demarcates normal from delayed gastric emptying.
8. The test subject's kPCD values are compared to the cutoff points of the reference range to qualitatively classify the subject as having normal or delayed gastric emptying. Generally, subjects classified as having normal gastric emptying typically display kPCD values that exceed time-specific cutoff points, reach a maximum kPCD value between 120-180 minutes, and then decline. In contrast, subjects classified as having delayed gastric emptying typically display kPCD values that are lower and rise continuously throughout the four-hour evaluation period. As a result, their highest kPCD value often occurs at four hours.
   Note that this exemplary test method can be performed in any desired setting, such as a clinical setting or a telehealth setting. In some cases, a clinician prepares the test meal whereas in other cases, the test subject prepares the test meal. Also, in some cases, the breath samples are analyzed in an outside lab by a lab technician. This exemplary method therefore is not limited to a particular setting and various steps can be performed in a single setting or across multiple settings.

### EXAMPLE 1

Applicant performed a percent binding experiment on two samples of the oatmeal-based test meal to determine the amount of ¹³C-Spirulina still bound to the food components after in-vitro partial digestion utilizing U.S.P gastric juice. In this experiment, the oatmeal-based test meal included 40 grams of oatmeal (e.g., instant oats), 177 mL of whole milk, 14 grams of cane sugar and 100 mg of ¹³C-Spirulina.

Applicant collected a portion (about 5 grams) of the oatmeal-based meal. This portion was assayed for ¹³C content prior to digestion. The rest underwent simulated in-vitro human gastric digestion using U.S.P gastric juice and then was also analyzed for ¹³C content. The ¹³C content of each half was measured using an Automated Nitrogen Carbon Analyzer Gas Chromatograph Isotope Ratio Mass Spectrometer to obtain a delta value (δ ¹³C). The resulting delta values before and after simulated digestion were then compared to provide a percent binding value. As shown in Table 1 below, Applicant found that the oatmeal-based test meal had a high percent binding value. Applicant also performed the same experiment on two samples of the egg-based meal. The values in Table 1 also show that the ¹³C-Spirulina binds to both the oatmeal-based meal and the egg-based meal.

TABLE 1
Percent Binding Values

| | Oatmeal-Based Meal | Egg-Based Meal |
|---|---|---|
| Sample 1 | 92.9% | 109.8% |
| Sample 2 | 98.6% | 109.4% |
| Average | 95.8% | 109.6% |

### EXAMPLE 2

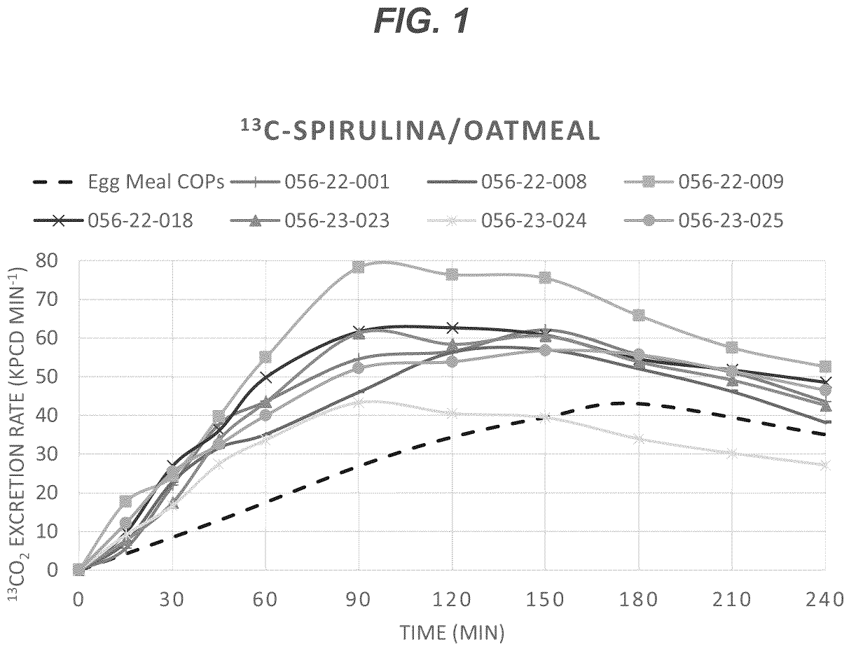

The GEBT method was performed on seven healthy subjects using the oatmeal-based test meal. Here too, in this experiment, the oatmeal-based test meal included 40 grams of oatmeal (e.g., instant oats), 177 mL of whole milk, 14 grams of cane sugar, 100 mg of ¹³C-Spirulina and 180 mL of water. The results are shown in Table 2 and plotted on a curve shown in FIG. 1.

TABLE 2
Individual kPCD results for Oatmeal-Based Meal
Subject IDs and ¹³CO₂ Excretion Rate (kPCD min⁻¹)

| | 056-22- | 056-22- | 056-22- | 056-22- | 056-23- | 056-23- | 056-23- | |
|---|---|---|---|---|---|---|---|---|
| Timepoint | 001 | 008 | 009 | 018 | 023 | 024 | 025 | Average |
| 0 | 0 | 0 | 0 | 0 | 0 | 0 | 0 | 0 |
| 15 | 5.6 | 7.2 | 17.6 | 9.7 | 7.7 | 9.1 | 12.1 | 9.9 |
| 30 | 21.9 | 22.8 | 24.2 | 26.9 | 17.4 | 16.6 | 25.3 | 22.2 |
| 45 | 37.7 | 31.6 | 39.7 | 36.1 | 33.7 | 27.4 | 32.4 | 34.1 |
| 60 | 43.6 | 35.1 | 54.9 | 49.8 | 43.4 | 33.6 | 39.9 | 42.9 |
| 90 | 54.6 | 46.0 | 78.2 | 61.6 | 61.2 | 43.3 | 52.1 | 56.7 |
| 120 | 56.6 | 56.3 | 76.3 | 62.6 | 58.3 | 40.5 | 53.8 | 57.8 |
| 150 | 62.1 | 57.0 | 75.4 | 60.7 | 60.4 | 39.4 | 56.7 | 58.8 |
| 180 | 55.7 | 52.0 | 65.7 | 54.5 | 53.7 | 33.9 | 55.6 | 53.0 |
| 210 | 51.1 | 46.1 | 57.4 | 51.7 | 49.0 | 30.2 | 51.3 | 48.1 |
| 240 | 43.5 | 38.1 | 52.5 | 48.5 | 42.5 | 27.1 | 46.4 | 42.7 |

### EXAMPLE 3

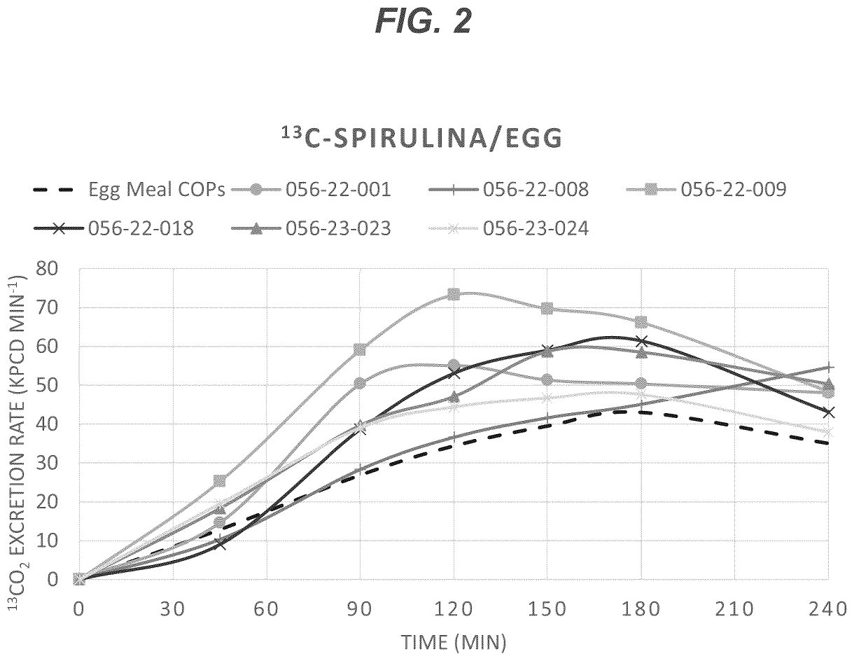

The GEBT method also was performed on six of the same healthy subjects in Example 2, using the egg-based test meal. In this experiment, the egg-based test meal included 27 grams of re-hydrated, pasteurized scrambled egg mix containing a dose of 43 mg of ¹³C (provided by approximately 100 mg of ¹³C-Spirulina), 6 saltine crackers, and 180 mL of water. The results are shown in Table 3 and plotted on a curve shown in FIG. 2.

TABLE 3
Individual kPCD results for Egg-Based Meal
Subject IDs and ¹³CO₂ Excretion Rate (kPCD min⁻¹)

| Time- | 056- | 056- | 056- | 056- | 056- | 056- | Aver- |
|---|---|---|---|---|---|---|---|
| point | 22-001 | 22-008 | 22-009 | 22-018 | 23-023 | 23-024 | age |
| 0 | 0 | 0 | 0 | 0 | 0 | 0 | 0 |
| 45 | 14.6 | 10.3 | 25.2 | 9.0 | 18.3 | 19.5 | 16.7 |
| 90 | 50.3 | 28.3 | 59.0 | 38.6 | 39.7 | 39.0 | 41.5 |
| 120 | 55.0 | 36.6 | 73.2 | 53.0 | 47.1 | 44.4 | 51.4 |
| 150 | 51.3 | 41.6 | 69.6 | 58.9 | 58.7 | 46.7 | 54.1 |
| 180 | 50.3 | 45.1 | 66.1 | 61.3 | 58.5 | 47.6 | 52.8 |
| 240 | 48.0 | 54.6 | 48.3 | 43.0 | 50.3 | 37.9 | 45.6 |

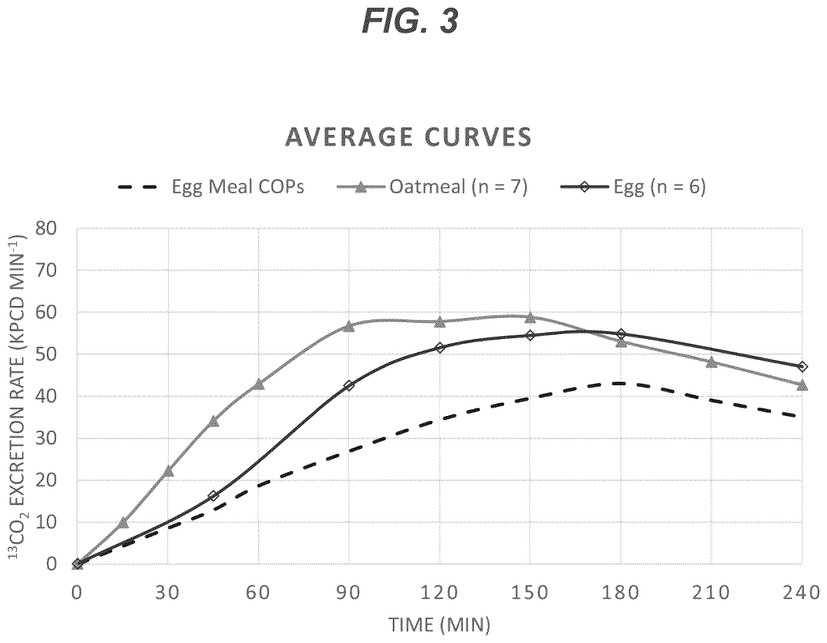

FIG. 3 illustrates a curve showing average kPCD values for the six healthy subjects that underwent the GEBT method for both the oatmeal-based meal and the egg-based meal. The results shown in Examples 2-3 show that the kPCD values surprisingly reflected similar rates and patterns of gastric emptying in subjects across both meals.

While some preferred embodiments of the invention have been described, it should be understood that various changes, adaptations and modifications may be made therein without departing from the spirit of the invention and the scope of the appended claims.

What is claimed is:

1. A breath test method, comprising the steps of:
obtaining a pre-meal baseline breath sample from a test subject;
providing a test meal comprising oatmeal and ¹³C-Spirulina, the ¹³C-Spirulina being bound to components of the oatmeal;
allowing the test subject to consume the test meal;
obtaining breath samples from the test subject at a series of time points after consumption of the test meal; and
analyzing the breath samples.

2. The breath test method of claim 1 wherein the series of time points includes 45, 90, 120, 150, 180 and 240 minute time points.

3. The breath test method of claim 1 wherein the ¹³C-Spirulina is present in an amount of from 50 mg to 200 mg.

4. The breath test method of claim 1 wherein the oatmeal is present in an amount of from 20 grams to 60 grams.

5. The breath test method of claim 1 further comprising whole milk.

6. The breath test method of claim 5 wherein the whole milk is present in an amount of from 90 mL to 270 mL.

7. The breath test method of claim 1 further comprising cane sugar.

8. The breath test method of claim 7 wherein the cane sugar is present in an amount of from 8 grams to 20 grams.

9. The breath test method of claim 1 further comprising water.

10. The breath test method of claim 9 wherein the water is present in an amount of from 120 mL to 240 mL.

11. The breath test method of claim 1 comprising oatmeal, whole milk, cane sugar and ¹³C-Spirulina.

12. The breath test method of claim 11 wherein the ¹³C-Spirulina is present in an amount of from 50 mg to 200 mg.

13. The breath test method of claim 11 wherein the oatmeal is present in an amount of from 20 grams to 60 grams.

14. The breath test method of claim 11 wherein the whole milk is present in an amount of from 90 mL to 270 mL.

15. The breath test method of claim 11 wherein the cane sugar is present in an amount of from 8 grams to 20 grams.

16. The breath test method of claim 11 comprising oatmeal in an amount of from 20 grams to 60 grams, whole milk in an amount from 90 mL to 270 mL, cane sugar in an amount of from 8 grams to 20 grams, and ¹³C-Spirulina in an amount of from 50 mg to 200 mg.

17. The breath test method of claim 11 further comprising water.

18. The breath test method of claim 17 wherein the water is present in an amount of from 120 mL to 240 mL.
